SEQUENCE LISTING (7 sequences)

SEQ ID NO: 1 - DNA, 3195 bp, synthetic construct
  source 1..3195: mol_type = other DNA
atgcccaccctcccccgcaccttcgacgacatccagtcccgcctcatcaacgccacctcccgcgtcgtccccatgcagcgccagatccagggcctcaagttcctcatgtccgccaagcgcaagaccttcggcccccgccgccccatgcccgagttcgtcgagacccccatccccgacgtcaacaccctcgccctcgaggacatcgacgtctccaaccccttcctctaccgccagggccagtggcgcgcctacttcaagcgcctccgcgacgaggcccccgtccactaccagaagaactcccccttcggccccttctggtccgtcacccgcttcgaggacatcctcttcgtcgacaagtcccacgacctcttctccgccgagccccagatcatcctcggcgacccccccgagggcctctccgtcgagatgttcatcgccatggacccccccaagcacgacgtccagcgctcctccgtccagggcgtcgtcgcccccaagaacctcaaggagatggagggcctcatccgctcccgcaccggcgacgtcctcgactccctccccaccgacaagcccttcaactgggtccccgccgtctccaaggagctcaccggccgcatgctcgccaccctcctcgacttcccctacgaggagcgccacaagctcgtcgagtggtccgaccgcctcgccggcgccgcctccgccaccggcggcgagttcgccgacgagaacgccatgttcgacgacgccgccgacatggcccgctccttctcccgcctctggcgcgacaaggaggcccgccgcgccgccggcgaggagcccggcttcgacctcatctccctcctccagtccaacaaggagaccaaggacctcatcaaccgccccatggagttcatcggcaacctcaccctcctcatcgtcggcggcaacgacaccacccgcaactccatgtccggcggcctcgtcgccatgaacgagttcccccgcgagttcgagaagctcaaggccaagcccgagctcatccccaacatggtctccgagatcatccgctggcagacccccctcgcctacatgcgccgcatcgccaagcaggacgtcgagctcggcggccagaccatcaagaagggcgaccgcgtcgtcatgtggtacgcctccggcaaccgcgacgagcgcaagttcgacaaccccgaccagttcatcatcgaccgcaaggacgcccgcaaccacatgtccttcggctacggcgtccaccgctgcatgggcaaccgcctcgccgagctccagctccgcatcctctgggaggagatcctcaagcgcttcgacaacatcgaggtcgtcgaggagcccgagcgcgtccagtccaacttcgtccgcggctactcccgcctcatggtcaagctcacccccaactccatgggcggcatcccctccccctcccgcgagcagtccgccaagaaggagcgcaagaccgtcgagaacgcccacaacacccccctcctcgtcctctacggctccaacatgggcaccgccgagggcaccgcccgcgacctcgccgacatcgccatgtccaagggcttcgccccccaggtcgccaccctcgactcccacgccggcaacctcccccgcgagggcgccgtcctcatcgtcaccgcctcctacaacggccacccccccgacaacgccaaggagttcgtcgactggctcgaccaggcctccgccgacgaggtcaagggcgtccgctactccgtcttcggctgcggcgacaagaactgggccaccacctaccagaaggtccccgccttcatcgacgagaccctcgccgccaagggcgccgagaacatcgccgagcgcggcgaggccgacgcctccgacgacttcgagggcacctacgaggagtggcgcgagcacatgtggtccgacctcgccgcctacttcaacctcgacatcgagaactccgaggagaacgcctccaccctctccctccagttcgtcgactccgccgccgacatgcccctcgccaagatgcaccgcgccttctccgccaacgtcgtcgcctccaaggagctccagaagcccggctccgcccgctccacccgccacctcgagatcgagctccccaaggaggcctcctaccaggagggcgaccacctcggcgtcatcccccgcaactacgagggcatcgtcaaccgcgtcgccacccgcttcggcctcgacgcctcccagcagatccgcctcgaggccgaggaggagaagctcgcccacctccccctcggcaagaccgtctccgtcgaggagctcctccagtacgtcgagctccaggaccccgtcacccgcacccagctccgcgccatggccgccaagaccgtctgccccccccacaaggtcgagctcgaggtcctcctcgagaagcaggcctacaaggagcaggtcctcgccaagcgcctcaccatgctcgagctcctcgagaagtaccccgcctgcgagatggagttctccgagttcatcgccctcctcccctccatgcgcccccgctactactccatctcctcctccccccgcgtcgacgagaagcaggcctccatcaccgtctccgtcgtctccggcgaggcctggtccggctacggcgagtacaagggcatcgcctccaactacctcgccaacctccaggagggcgacaccatcacctgcttcgtctccaccccccagtccggcttcaccctccccaagggccccgagacccccctcatcatggtcggccccggcaccggcgtcgcccccttccgcggcttcgtccaggcccgcaagcagctcaaggagcagggccagtccctcggcgaggcccacctctacttcggctgccgctccccccacgaggactacctctaccagaaggagctcgagaacgcccagaacgagggcatcatcaccctccacaccgccttctcccgcgtccccaaccagcccaagacctacgtccagcacgtcatggagcaggacggcaagaagctcatcgagctcctcgaccagggcgcccacttctacatctgcggcgacggctcccagatggcccccgacgtcgaggccaccctcatgaagtcctacgccgaggtccaccaggtctccgaggccgacgcccgcctctggctccagcagctcgaggagaagggccgctacgccaaggacgtctgggccggctaa

SEQ ID NO: 2 - DNA, 1266 bp, synthetic construct
  source 1..1266: mol_type = other DNA
atggtccggattcttcccataattttgagcgccctatcttcgaaattagtggcgagtacaatattgcattcatccatacactcagtgccatctggaggcgaaatcatatctgcagaagatcttaaagaacttgaaatttcagggaattcgatctgcgttgataatcgttgctatcctaagatatttgaaccaagacacgattggcagcccatactgccaggtcaagaactccccggtggtttggacattagaataaacatggacacaggtttaaaagaggcaaaactaaatgatgagaagaatgtcggtgataatggtagccatgagttaattgtatcttcagaagacatgaaagcatcgcctggtgactatgaattttccagtgatttcaaagaaatgagaaacatcatagattctaacccgactttatcttcacaggacattgccagattggaggatagttttgatagaataatggaatttgcgcatgattacaagcacggctacaaaattattacccatgaattcgccctcttggccaaccttagtctcaatgaaaatttgccgttaacattgagagagctcagtactagagtcattaccagctgcttgagaaacaatcctcctgtagtcgagttcattaatgaaagttttccaaattttaaaagcaaaatcatggccgctctgtcaaatttgaatgattctaaccacagatcctctaatatcctaataaaaagatacttgtccattttaaacgaattacctgtcacatccgaagatcttcctatatactctacggttgttttacaaaatgtatatgaaagaaacaacaaggacaaacagttacaaataaaagtcctggagttgatcagcaaaattttgaaggccgacatgtacgaaaatgacgatacaaatctaattttgttcaaaagaaatgctgagaattggtcgtcaaatctgcaagagtgggcaaacgagttccaagagatggtccagaacaaaagtatagatgaactacatacaagaacgttttttgacaccctttacaacttgaagaaaattttcaaaagtgacatcacgatcaacaaagggtttttgaattggttagcgcaacaatgtaaagccaggcaatctaacttggacaatgggctccaagagagagatactgaacaagactcatttgataagaaacttatcgacagcagacacttgatctttggcaaccccatggctcatagaataaaaaatttcagagatgaactctga

SEQ ID NO: 3 - DNA, 675 bp, synthetic construct
  source 1..675: mol_type = other DNA
atgaagcttcaatttttttcctttattaccttatttgcttgtctcttcacaacagccatttttgcgaaagaggacacggcagaagatcctgagatcacacacaaggtgtactttgacattaatcacggtgataaacaaattggtagaattgttatgggattgtatggtctcaccacaccccaaaccgttgaaaacttttaccagttgaccatttccagggaccctaagatgggttatttgaactctatcttccatcgcgttattcctaacttcatgattcaaggtggcgatttcactcacagatcaggtattgggggtaagtctatcttcggaaacacgttcaaagatgagaattttgatgtcaaacatgacaaaccaggcagattgtctatggccaatcgtggtaaaaacaccaacggatcccaatttttcatcaccaccgtcccatgcccatggttggacggtaagcacgttgtctttggagaagtcttggatggtatggacgtagttcactacattgaaaacgttaagaccgacagtagaaatatgcctgtaaaggaagttattattgtggaaagtggtgaactggaaactgttcctttggacaataaagacgccgccaagctacaggaagagatcaaagcggaagctagcgaagcagcccacgatgaactc

SEQ ID NO: 4 - DNA, 27 bp, synthetic construct
  source 1..27: mol_type = other DNA
cggtttgtttgggtttgggtttgggtt

SEQ ID NO: 5 - DNA, 27 bp, synthetic construct
  source 1..27: mol_type = other DNA
ggcttgccttacccttacccttaccct

SEQ ID NO: 6 - DNA, 23 bp, synthetic construct
  source 1..23: mol_type = other DNA
cagcaagtgggaaggtgtaatcc

SEQ ID NO: 7 - DNA, 24 bp, synthetic construct
  source 1..24: mol_type = other DNA
cccattctatcatcaacggtacaa

(19) United States
(12) Patent Application Publication
(10) Pub. No.: US 2026/0160767 A1
(43) Pub. Date: Jun. 11, 2026

(54) Biosynthetic Method for Preparing 10-Hydroxy-2-Decenoic Acid and Use Thereof in Skin Care

(71) Applicant: SHANDONG FREDA BIOTECH CO., LTD., Jinan (CN)

(72) Inventors: Suzhen YANG, Jinan (CN); Yan LI, Jinan (CN); Chunying YUAN, Jinan (CN); Tingting HAN, Jinan (CN); Peipei XU, Jinan (CN); Chunming GAO, Jinan (CN); Ruiming WANG, Jinan (CN); Jing SU, Jinan (CN); Kuankuan GAO, Jinan (CN); Yurong CHEN, Jinan (CN); Shanshan JIANG, Jinan (CN); Fei LIU, Jinan (CN); Sanling LIU, Jinan (CN); Xinyu MAO, Jinan (CN)

(21) Appl. No.: 19/537,664

(22) Filed: Feb. 12, 2026

Related U.S. Application Data
(63) Continuation of application No. PCT/CN2024/113117, filed on Aug. 19, 2024.

(30) Foreign Application Priority Data
Aug. 21, 2023 (CN) 2023110480432

(51) Int. Cl. G01N 33/68; G01N 33/50

(52) U.S. Cl. CPC G01N 33/6848; G01N 33/5044; G01N 2405/00

(57) ABSTRACT

The present disclosure relates to genetic engineering and biosynthetic technologies, and provides a biosynthetic method for preparing 10-HDA and use thereof in skin care. An engineered yeast strain is fermented in a culture medium containing trans-2-decenoic acid to produce 10-HDA. In vitro evaluations using a 3D epidermal model and skin fibroblasts indicate that 10-HDA can increase stratum corneum lipid parameters including ceramides and fatty acids, reduce UV-A-induced DNA damage markers, improve mitochondrial function by decreasing mitochondrial ROS and early apoptosis and increasing ATP and NADPH, and increase relative telomere length under oxidative stress, thereby providing potential benefits for skin barrier repair and anti-aging skin care products.

### SEQUENCE LISTING STATEMENT

The present disclosure contains a Sequence Listing submitted in electronic form (ST.26 XML), the file name of which is “PCT2024043US-Sequence-Listing”, with a creation date of “Feb. 9, 2026” and a file size of “12,412 bytes”. The contents of the electronic Sequence Listing are incorporated herein by reference in their entirety.

### CROSS-REFERENCE TO RELATED APPLICATIONS

The present disclosure claims priority to a Chinese patent application filed with the China National Intellectual Property Administration on Aug. 21, 2023, having Application No. 202311048043.2 and entitled “Biosynthetic Method for Preparing 10-Hydroxy-2-Decenoic Acid and Use Thereof in Skin Care”, the entire contents of which are incorporated herein by reference in their entirety and for all purposes.

### TECHNICAL FIELD

The present disclosure relates to the fields of genetic engineering and biosynthetic technologies, and in particular relates to a biosynthetic method for preparing 10-hydroxy-2-decenoic acid and use thereof in skin care.

### BACKGROUND OF THE INVENTION

The information disclosed in this Background section is provided solely to increase understanding of the general background of the present disclosure and is not necessarily an admission, nor should it be construed as any form of suggestion, that such information constitutes prior art that is known to a person of ordinary skill in the art.

Royal jelly acid, namely 10-hydroxy-2-decenoic acid (10-HDA), is an unsaturated fatty acid present in royal jelly. The source of 10-HDA is highly specific, and it naturally occurs only in royal jelly, where its content is relatively low, typically about 1.4% to 2.4%. Studies on the biological activities of 10-HDA have shown that, similar to royal jelly, 10-HDA exhibits significant health-promoting and therapeutic effects, including antibacterial, antitumor, and antioxidant activities, enhancement of immune function, regulation of blood glucose levels, among others.

At present, domestic production of 10-HDA mainly relies on physical extraction methods and chemical synthesis methods. The extraction cost is high and cannot meet large-scale demand. Currently, physical extraction from royal jelly is generally used. Because the amount of royal jelly is limited, this extraction method is not suitable for large-scale production; it also uses large quantities of organic solvents and is relatively costly. In terms of chemical preparation, methods such as addition-hydrolysis of α-olefins with carbon tetrachloride, α-dehalogenation of carboxylic acids, and the Knoevenagel condensation are commonly used. However, most chemical synthesis methods require harsh reaction conditions; except for the condensation method, yields are relatively low; the resulting double bond is a cis/trans mixture, which is difficult to isolate and purify. At present, biosynthetic preparation of 10-HDA is still at a research stage and the yield is unstable. There is an urgent need to obtain engineered strains with stable functionality to enable batch production of 10-HDA.

The skin barrier forms a protective layer between the “inside” and the “outside” and prevents external harm. Epidermal lipids such as ceramides, fatty acids, triglycerides, and cholesterol are essential components for the formation and maintenance of the epidermal barrier function. Fatty acids account for about 15%-20% of the total lipid weight in the stratum corneum and play an important role in maintaining the structural stability of the human stratum corneum and in repairing epidermal barrier damage. A reduction in fatty acids can impair the skin barrier function and lead to various skin inflammations. Studies have shown that the lipid content in aged skin decreases significantly, which is also an important reason for the decline of barrier function in aged skin. In the skin, reactive oxygen species (ROS) can react with the protective lipid bilayer in the stratum corneum to form lipid hydroperoxides, which may weaken the skin barrier function. Therefore, maintaining the levels of stratum corneum cellular fatty acids and ceramides, and reducing lipid peroxidation, are effective means to repair a damaged skin barrier. Under external environmental stimuli, a reduction in cell lifespan caused by early cellular senescence is mainly attributable to nuclear and DNA damage. Abnormal nuclear morphology impairs nuclear functions, including altering histone modification patterns, abnormal chromatin regeneration, impaired nuclear transport, delayed DNA repair responses, and shortening of nuclear telomere length. Accordingly, repairing damaged DNA and extending nuclear telomere length are key to resisting cellular senescence. In addition, during normal cellular life activities, mitochondria are the power source for all life activities, providing more than 90% of cellular energy and deeply regulating activities such as energy metabolism, ROS and free radicals, oxidative stress, and inflammation. While utilizing oxygen molecules, mitochondria are also continuously damaged by free radicals, leading to mitochondrial dysfunction and skin aging. Therefore, skin aging induced by external stimuli may be counteracted through approaches such as enhancing the skin lipid barrier, reducing mitochondrial damage, repairing cellular DNA, and slowing nuclear telomere shortening.

### SUMMARY OF THE INVENTION

In view of the above prior art, the present disclosure is directed to providing a biosynthetic method for preparing 10-HDA and use thereof in skin care. The present disclosure is based on producing 10-HDA by fermenting an engineered yeast strain using a culture medium containing trans-2-decenoic acid. As evaluated at the levels of skin cells and skin models, 10-HDA can promote the synthesis of stratum corneum fatty acids and ceramides in aged skin to repair the skin barrier. 10-HDA can also promote the synthesis of mitochondrial ATP and NADPH in skin fibroblasts, reduce mitochondrial reactive oxygen species, improve mitochondrial dysfunction, and inhibit apoptosis. Further experiments show that 10-HDA can repair DNA damage in fibroblasts under ultraviolet irradiation and can slow telomere shortening caused by cell division and replication, thereby strengthening protection of chromosome ends. Based on the above findings, the present disclosure is provided.

To achieve the above technical objectives, the technical solutions of the present disclosure are as follows.

In a first aspect, the present disclosure provides a method for preparing 10-HDA based on biosynthesis, the method comprising:
fermenting an engineered yeast strain comprising at least a CYP153A33(M228L)-CPR_{BM3} fusion gene using a culture medium containing trans-2-decenoic acid to produce 10-HDA.

A yeast engineered strain containing at least a CYP153A33(M228L)-CPR_{BM3} fusion gene is fermented in a medium containing trans-2-decenoic acid to produce royal jelly acid. In the present disclosure, the CYP153A33(M228L)-CPR_{BM3} fusion gene refers to a fusion gene formed by fusing the coding region of CYP153A33 with the coding region of CPR_{BM3}. The CYP153A33 comprises an M228L amino acid substitution, i.e., the amino acid at position 228 of the CYP153A33 protein is substituted from methionine (M) to leucine (L).

In a specific embodiment, the nucleotide sequence of the CYP153A33(M228L)-CPR_{BM3} fusion gene is shown in SEQ ID NO: 1. The fusion gene is codon-optimized to facilitate expression in Saccharomyces cerevisiae (S. cerevisiae). Experimental results demonstrate that an engineered yeast strain expressing the above CYP153A33(M228L)-CPR_{BM3} fusion gene can react with the substrate trans-2-decenoic acid to obtain 10-HDA, with a yield reaching 190 mg/L.

In some embodiments, the engineered yeast strain can further comprise an auxiliary gene sil1p and/or an auxiliary gene cpr5p.

In some embodiments, the nucleotide sequence of the auxiliary gene sil1p is as shown in SEQ ID NO: 2, and the nucleotide sequence of the auxiliary gene cpr5p is as shown in SEQ ID NO: 3. By introducing the auxiliary gene sil1p and cpr5p, the capability of the engineered yeast strain to produce 10-HDA can be further improved.

The method can further comprise any one or more of separation, purification, and drying of 10-HDA. Accordingly, 10-HDA can be provided as a liquid preparation or a solid preparation (e.g., granules, powders).

In a second aspect, the present disclosure provides use of the above method and/or 10-HDA produced by the above method in any one or more of the following:
(a) increasing contents of ceramides and fatty acids in an epidermal layer to repair a skin barrier, or preparing a product for increasing contents of ceramides and fatty acids in an epidermal layer and repairing a skin barrier;
(b) repairing DNA damage of dermal fibroblasts, or preparing a product for repairing DNA damage of dermal fibroblasts;
(c) improving mitochondrial function, enhancing cellular function, and promoting metabolism, or preparing a product for improving mitochondrial function, enhancing cellular function, and promoting metabolism; and
(d) delaying telomere shortening caused by cell division and replication and protecting structural integrity of chromosome ends, or preparing a product for delaying telomere shortening caused by cell division and replication and protecting structural integrity of chromosome ends.

In some embodiments, the present disclosure provides an in vitro method for improving at least one skin-related biomarker in a skin-related in vitro system. The in vitro method comprises contacting the skin-related in vitro system with 10-HDA and determining a level or change of the at least one skin-related biomarker. The skin-related in vitro system can be selected from a 3D epidermal model and skin fibroblasts.

In some embodiments, when the skin-related in vitro system is the 3D epidermal model, the at least one skin-related biomarker is a stratum corneum lipid level; and, relative to an untreated control system not contacted with 10-HDA, the contacting causes the stratum corneum lipid level to increase.

In some embodiments, when the skin-related in vitro system is the skin fibroblasts, the at least one skin-related biomarker is selected from: (A) a DNA damage marker level, (B) a mitochondrial function metric, and (C) a relative telomere length; and, relative to an untreated control system not contacted with 10-HDA, the contacting causes at least one of: (1) the DNA damage marker level to decrease; (2) the mitochondrial function metric to improve, the improvement comprising a decrease in mitochondrial ROS level and/or early apoptosis rate, and/or an increase in mitochondrial ATP content and/or cellular NADPH content; and (3) the relative telomere length to increase.

The beneficial technical effects of one or more of the above technical solutions include:

1) compared with physical extraction and chemical synthesis methods for preparing 10-HDA, the above technical solutions provide a method for preparing 10-HDA via biosynthesis, which is natural, green, and safe. In addition, the above technical solutions, for the first time, codon-optimize and express the CYP153A33(M228L)-CPR_{BM3} fusion gene in S. cerevisiae. Recombinant expression of CYP153A33(M228L)-CPR_{BM3} can react with the substrate trans-2-decenoic acid to obtain 10-HDA, with a yield reaching 190 mg/L, which is superior to the yield of 10-HDA obtained from decenoic acid catalyzed by the recombinant protein CYP539A7*-F0CPR* reported in CN202211449654.3. Further, the above technical solutions co-transform and co-express the CYP153A33(M228L)-CPR_{BM3} fusion gene with the auxiliary protein gene sil1p and the auxiliary protein gene cpr5p in S. cerevisiae, which can effectively increase the yield of 10-HDA in the engineered yeast strain. With connection of the auxiliary protein gene sil1p to the protein, the 10-HDA yield can reach 280 mg/L; and with connection of the auxiliary protein gene cpr5p to the fusion enzyme gene, the 10-HDA yield can reach 246 mg/L, both of which are superior to the yield of 10-HDA obtained from trans-2-decenoic acid catalyzed by the recombinant strain constructed by connecting CYP539A7*-F0CPR* with an auxiliary protein as reported in CN202211449654.3. In particular, the time for preparing 10-HDA using the engineered S. cerevisiae strain constructed by the above technical solutions is significantly shorter than the reaction time reported in CN202211449654.3. The reaction time of the engineered strain in CN202211449654.3 is 48 hours. In the above technical solutions, the engineered strain harboring a co-expression plasmid of CYP153A33(M228L)-CPR_{BM3} and the auxiliary protein gene sil1p in S. cerevisiae can reach the maximum conversion rate and product conversion amount after reacting with 0.5 g/L decenoic acid for 36 hours, thereby achieving excellent technical effects.

2) For 10-HDA prepared by the above technical solutions, in a 3D epidermal model, 10-HDA treatment can significantly increase stratum corneum lipid-related indices: total ceramide increases by 35.37%, ceramide/protein (i.e., total ceramide normalized by sample protein amount) increases by 47.64%; total fatty acid increases by 27.51%, and fatty acid/protein (i.e., total fatty acid normalized by sample protein amount) increases by 39.0% (each relative to an untreated control system). These results indicate that, in the 3D epidermal model, 10-HDA can increase barrier-related lipid levels, suggesting potential to improve barrier-related parameters.

3) For 10-HDA prepared by the above technical solutions, in a fibroblast model, contents of 8-hydroxy-2′-deoxyguanosine and γ-H2AX protein are reduced, with inhibition rates of 45.09% and 47.25%, respectively, thereby inhibiting DNA damage and preventing functional abnormalities caused by accumulation of damage. 10-HDA can reduce mitochondrial reactive oxygen species and the early apoptosis rate in cells, with inhibition rates of 70.71% and 12.74%, respectively, and can increase contents of mitochondrial adenosine triphosphate (ATP) and nicotinamide adenine dinucleotide phosphate (NADPH), with increases of 56.23% and 15.13%, respectively, thereby protecting mitochondria from peroxidative damage and maintaining intracellular energy supply and substance metabolism. 10-HDA can also extend telomere length, with the relative T/S (TL) value increased by 31%, thereby effectively delaying telomere shortening caused by cell division and replication in the senescence process, strengthening structural protection of chromosome ends, preventing degradation or fusion of chromosome ends, preventing cells from losing proliferative activity and entering senescence, and further preventing skin aging.

In summary, 10-HDA prepared by the above technical solutions and products thereof have broad application prospects in development of functional cosmetics, particularly skin care products, for skin repair and anti-aging.

### BRIEF DESCRIPTION OF DRAWINGS

The accompanying drawings, which constitute a part of this specification, provide further understanding of the present disclosure. The schematic embodiments and descriptions thereof are used to explain the present disclosure and do not constitute an improper limitation of the present disclosure.

FIG. 1 is an agarose gel electrophoresis image of PCR amplification products in Example 1 of the present disclosure; lane M is a marker; lanes 1˜4 are PCR amplification product samples of CYP153A33(M228L)-CPR_{BM3}; lanes 5-6 are PCR amplification product samples of the auxiliary protein gene sil1p; and lanes 7-8 are PCR amplification product samples of the auxiliary protein gene cpr5p.

FIG. 2 is a schematic diagram of construction of the plasmid pESC-URA-CYP153A33(M228L)-CPR_{BM3} in Example 1 of the present disclosure.

FIG. 3 is a schematic diagram of construction of the plasmid pESC-URA-CYP153A33(M228L)-CPR_{BM3}-sil1p in Example 1 of the present disclosure.

FIG. 4 is a schematic diagram of construction of the plasmid pESC-URA-CYP153A33(M228L)-CPR_{BM3}-cpr5p in Example 1 of the present disclosure.

FIG. 5 is an agarose gel electrophoresis image of colony-PCR products in Example 1 of the present disclosure; lane M is a marker; lanes 1˜4 are colony-PCR amplification product samples from different single colonies of E. coli DH5α/pESC-URA-CYP153A33(M228L)-CPR_{BM3}; lanes 5-8 are colony-PCR amplification product samples from different single colonies of E. coli DH5α/pESC-URA-CYP153A33(M228L)-CPR_{BM3}-sil1p; and lanes 9-12 are colony-PCR amplification product samples from different single colonies of E. coli DH5α/pESC-URA-CYP153A33(M228L)-CPR_{BM3}-cpr5p.

FIG. 6 is a graph of 10-HDA yields after a 48-hour reaction under different engineered strains in Example 1 of the present disclosure; from left to right are: contents of trans-2-decenoic acid and 10-HDA for the engineered S. cerevisiae strain BY4741/pESC-URA-CYP153A33(M228L)-CPR_{BM3}; contents of trans-2-decenoic acid and 10-HDA for the engineered S. cerevisiae strain BY4741/pESC-URA-CYP153A33(M228L)-CPR_{BM3}-sil1p; and contents of trans-2-decenoic acid and 10-HDA for the engineered S. cerevisiae strain BY4741/pESC-URA-CYP153A33(M228L)-CPR_{BM3}-cpr5p.

FIG. 7 is a time-course line chart of yields for the engineered S. cerevisiae strain BY4741/pESC-URA-CYP153A33(M228L)-CPR_{BM3}-sil1p and the engineered S. cerevisiae strain BY4741/pESC-URA-CYP539A7-F0CPR-sil1p in Example 1 of the present disclosure.

FIG. 8 is a graph showing regulation of ceramide and fatty acid expression in a 3D epidermal model by 10-HDA in Example 2 of the present disclosure.

FIG. 9 is a graph showing detection results of the DNA damage products 8-hydroxy-2′-deoxyguanosine and γ-H2AX in fibroblasts treated with 10-HDA in Example 3 of the present disclosure.

FIG. 10 shows immunofluorescence staining results of the DNA damage product γ-H2AX in fibroblasts treated with 10-HDA in Example 3 of the present disclosure.

FIG. 11 shows detection results of mitochondrial function-related indices in fibroblasts treated with 10-HDA in Example 4 of the present disclosure; the indices include: the mean value of relative total integrated optical density (IOD) of ROS, mitochondrial ATP content, early apoptosis rate, and NADPH content.

FIG. 12 shows staining results of reactive oxygen species in fibroblasts treated with 10-HDA in Example 4 of the present disclosure.

FIG. 13 shows the effect of 10-HDA on telomere length in fibroblasts in Example 5 of the present disclosure.

FIG. 14 shows changes of red-area parameters on day 14 and day 28 after use of a skin care lotion by subjects in Example 6 of the present disclosure.

FIG. 15 shows changes of wrinkles on day 14 and day 28 after use of a skin care lotion by subjects in Example 6 of the present disclosure.

### DETAILED DESCRIPTION

It should be noted that the following detailed description is exemplary and is intended to provide further explanation of the present disclosure. Unless otherwise indicated, all technical and scientific terms used herein have the same meanings as commonly understood by a person of ordinary skill in the art to which the present disclosure pertains.

It should be understood that the terms used herein are only for describing particular embodiments and are not intended to limit the exemplary embodiments of the present disclosure. As used herein, unless expressly indicated otherwise by the context, the singular forms are intended to include the plural forms. It should also be understood that, when the terms “comprise” and/or “include” are used in this specification, they specify the presence of stated features, steps, operations, devices, components, and/or combinations thereof.

The present disclosure is further described below in conjunction with specific examples, which are provided only for illustration and do not limit the content of the present disclosure. If specific experimental conditions are not indicated in the examples, the experiments are generally conducted under conventional conditions or under conditions recommended by reagent suppliers. Unless otherwise specified, the reagents, consumables, etc. used in the examples are commercially available.

In a typical embodiment of the present disclosure, a biosynthetic method for preparing 10-HDA is provided, the method comprising:

fermenting an engineered yeast strain comprising at least a CYP153A33(M228L)-CPR_{BM3} fusion gene using a culture medium containing trans-2-decenoic acid to produce 10-HDA.

In some embodiments, the nucleotide sequence of the CYP153A33(M228L)-CPR_{BM3} fusion gene is as shown in SEQ ID NO: 1. The fusion gene is codon-optimized to facilitate expression in S. cerevisiae. Experimental results demonstrate that an engineered yeast strain expressing the above CYP153A33(M228L)-CPR_{BM3} fusion gene can react with the substrate trans-2-decenoic acid to obtain 10-HDA, with a yield reaching 190 mg/L.

In some embodiments, the engineered yeast strain can further comprise an auxiliary gene sil1p and/or an auxiliary gene cpr5p.

In some embodiments, the nucleotide sequence of the auxiliary gene sil1p is as shown in SEQ ID NO: 2, and the nucleotide sequence of the auxiliary gene cpr5p is as shown in SEQ ID NO: 3. By introducing the auxiliary gene sil1p and cpr5p, the capability of the engineered yeast strain to produce 10-HDA can be further improved.

In the present disclosure, the original starting strain of the engineered yeast strain is S. cerevisiae. In some embodiments, the original starting strain can be S. cerevisiae BY4741. S. cerevisiae BY4741 is an auxotrophic strain deficient in methionine, leucine, histidine, and uracil. It is a commonly used laboratory strain and is widely used in studies of, for example, sodium/potassium ion balance, cellular salt resistance, uptake of various metal ions, heavy-metal toxicity, effects of various sugars and carbon sources on growth of eukaryotic cells, and uptake and transport of peroxides and superoxides, and it is commercially available.

In some embodiments, the engineered yeast strain is constructed as follows: introducing a recombinant expression vector comprising the CYP153A33(M228L)-CPR_{BM3} fusion gene into the original starting strain.

The recombinant expression vector can be obtained by operably linking the above CYP153A33(M228L)-CPR_{BM3} fusion gene to an expression vector. The expression vector can be any one or more of a viral vector, a plasmid, a phage, a cosmid, or an artificial chromosome. In another embodiment of the present disclosure, the expression vector is a plasmid; more specifically, the expression vector can be a pESC-URA plasmid.

In some embodiments, the recombinant expression vector can further be linked to the auxiliary gene sil1p and/or the auxiliary gene cpr5p.

In some embodiments, the biosynthetic method for preparing 10-HDA specifically comprises:

S1. Inoculating the engineered yeast strain into a uracil-dropout seed medium at pH 5.0-6.0, and culturing overnight with shaking at 25-35° C.; inoculating a seed culture into a uracil-dropout fermentation medium with an initial OD₆₀₀ of 0.3-0.5; inducing by shaking at 25-35° C. until the culture reaches an OD₆₀₀ of 1.0-1.2; and collecting the cells.

S2. Resuspending the cells collected in step S1 in the uracil-dropout fermentation medium of step S1; adding trans-2-decenoic acid to the medium; and fermenting to obtain 10-HDA.

In step S1, the uracil-dropout seed medium comprises a basal medium YNB, glucose at 1.5%-2.5% (w/v), and a uracil-dropout amino-acid mixture at 1.0-1.5 g/L.

The uracil-dropout fermentation medium comprises a basal medium YNB, galactose at 3.5%-4.5% (w/v), 2 mM 5-aminolevulinic acid (5-ALA), and a uracil-dropout amino-acid mixture at 1.0-1.5 g/L.

In step S2, the cells are collected by centrifuging the culture at 3500-4000 rpm for 10-15 min to collect the pellet.

Fermentation conditions include culturing at 25-35° C. for 24-72 hours, for example, 24, 36, 48, 60, or 72 hours. Studies have shown that the engineered yeast strain of the present disclosure can reach a maximum conversion rate and product conversion amount after reacting with 0.5 g/L trans-2-decenoic acid for 36 hours, which is more favorable for practical industrial production.

The concentration of trans-2-decenoic acid added to the culture medium is 0.1-2.0 g/L, and preferably 0.5-1.0 g/L.

In some embodiments, the method can further comprise any one or more of separation, purification, and drying of 10-HDA. Accordingly, 10-HDA can be provided as a liquid preparation or a solid preparation (e.g., granules, powders), without limitation.

In another embodiment of the present disclosure, use of the above method and/or 10-HDA produced by the above method is provided in any one or more of the following:
(a) increasing contents of ceramides and fatty acids in an epidermal layer to repair a skin barrier, or preparing a product for increasing contents of ceramides and fatty acids in an epidermal layer and repairing a skin barrier;
(b) repairing DNA damage of dermal fibroblasts, or preparing a product for repairing DNA damage of dermal fibroblasts;
(c) improving mitochondrial function, enhancing cellular function, and promoting metabolism, or preparing a product for improving mitochondrial function, enhancing cellular function, and promoting metabolism; and
(d) delaying telomere shortening caused by cell division and replication and protecting structural integrity of chromosome ends, or preparing a product for delaying telomere shortening caused by cell division and replication and protecting structural integrity of chromosome ends.

The product can be a food, a pharmaceutical, or a cosmetic. In some embodiments, the cosmetic is a skin care product, thereby effectively repairing the skin and providing anti-aging effects.

The cosmetic can further comprise other raw material ingredients permitted in the cosmetic field, including but not limited to emulsifiers, emollients, humectants, thickeners, and the like. A person of ordinary skill in the art can select and add such ingredients as appropriate. Correspondingly, the cosmetic dosage form may be a cream, a lotion, an aqueous formulation, a gel, a powder, an aerosol, a patch, a mask, among others, and is not particularly limited thereto.

In some embodiments, an in vitro method is provided for improving at least one skin-related biomarker in a skin-related in vitro system. The in vitro method comprises contacting the skin-related in vitro system with 10-HDA and determining a level or change of the at least one skin-related biomarker. The skin-related in vitro system can be selected from a 3D epidermal model and skin fibroblasts. Examples 2-5 exemplarily provide experimental designs and detection methods of the above in vitro method.

In some embodiments, when the skin-related in vitro system is the 3D epidermal model, the at least one skin-related biomarker is a stratum corneum lipid level; and, relative to an untreated control system not contacted with 10-HDA, the contacting causes the stratum corneum lipid level to increase.

In some embodiments, when the skin-related in vitro system is the skin fibroblasts, the at least one skin-related biomarker is selected from: (A) a DNA damage marker level, (B) a mitochondrial function metric, and (C) a relative telomere length; and, relative to an untreated control system not contacted with 10-HDA, the contacting causes at least one of: (1) the DNA damage marker level to decrease; (2) the mitochondrial function metric to improve, the improvement comprising a decrease in mitochondrial ROS level and/or early apoptosis rate, and/or an increase in mitochondrial ATP content and/or cellular NADPH content; and (3) the relative telomere length to increase.

As used herein, an “untreated control system” refers to a control system that is not contacted with 10-HDA under the corresponding experimental conditions. Except for the absence of 10-HDA, the untreated control system is kept consistent with a corresponding sample system in terms of stress treatment (if any), culture conditions, treatment sequence, and detection steps.

As used herein, “improvement/improved” in an in vitro system can be manifested as at least one directional change: an increase in stratum corneum lipid level; a decrease in DNA damage marker level; improvement of a mitochondrial function metric including a decrease in mitochondrial ROS level and/or early apoptosis rate, and/or an increase in mitochondrial ATP content and/or cellular NADPH content; and/or an increase in relative telomere length. Such determination can be made using the detection methods described in the Examples herein.

Unless otherwise indicated, in Examples 2-5, “increase/decrease” and the corresponding percentage change are determined with reference to a corresponding untreated control system. For experiments involving UV-A irradiation or H₂O₂ stimulation, the untreated control system refers to a control system subjected to the same stress conditions but not contacted with 10-HDA.

In some embodiments, the skin-related in vitro system is a 3D epidermal model. The in vitro method comprises contacting the 3D epidermal model with 10-HDA and determining a stratum corneum lipid level. The stratum corneum lipid level can include one or more of: total ceramide, ceramide/protein, total fatty acid, and fatty acid/protein. Relative to an untreated control 3D epidermal model system not contacted with 10-HDA, the contacting causes the stratum corneum lipid level to increase.

In some embodiments, lipids are extracted from stratum corneum samples, and liquid chromatography-mass spectrometry (LC-MS) is used to quantify the stratum corneum lipid level.

In some embodiments, when the 3D epidermal model is contacted with 10-HDA, the final concentration of 10-HDA in the 3D epidermal model culture system is 0.0625 mg/mL.

In some embodiments (e.g., Example 2), relative to an untreated control 3D epidermal model system not contacted with 10-HDA: (i) total ceramide increases by 35.37% and/or ceramide/protein increases by 47.64%; and/or (ii) total fatty acid increases by 27.51% and/or fatty acid/protein increases by 39.0%.

In some embodiments, the skin-related in vitro system is skin fibroblasts. The skin fibroblasts are subjected to UV-A irradiation at a dose of 30 J/cm² and cultured for an additional 24 hours after irradiation; the final concentration of 10-HDA in the skin fibroblast culture system is 0.25 mg/mL. A DNA damage marker level is determined, the DNA damage marker level including an 8-OHdG level and/or a γ-H2AX level. Relative to an untreated control skin fibroblast system subjected to the same UV-A irradiation but not contacted with 10-HDA, the DNA damage marker level decreases.

In some embodiments (e.g., Example 3), relative to an untreated control skin fibroblast system subjected to the same UV-A irradiation but not contacted with 10-HDA, the 8-OHdG level decreases by 45.09%.

In some embodiments (e.g., Example 3), relative to an untreated control skin fibroblast system subjected to the same UV-A irradiation but not contacted with 10-HDA, the γ-H2AX level decreases by 47.25%, and the γ-H2AX level is measured by γ-H2AX immunofluorescence.

In some embodiments, the skin-related in vitro system is skin fibroblasts. The skin fibroblasts are subjected to UV-A irradiation at a dose of 30 J/cm² and cultured for an additional 24 hours after irradiation; the final concentration of 10-HDA in the skin fibroblast culture system is 0.25 mg/mL. A mitochondrial function metric is determined, the mitochondrial function metric including one or more of: mitochondrial ROS level, mitochondrial ATP content, cellular NADPH content, and early apoptosis rate. Relative to an untreated control skin fibroblast system subjected to the same UV-A irradiation but not contacted with 10-HDA, the mitochondrial function metric is improved.

In some embodiments (e.g., Example 4), relative to an untreated control skin fibroblast system subjected to the same UV-A irradiation but not contacted with 10-HDA: mitochondrial ROS level decreases by 70.71%, and/or mitochondrial ATP content increases by 56.23%, and/or cellular NADPH content increases by 15.13%, and/or early apoptosis rate decreases by 12.74%.

In some embodiments, the skin-related in vitro system is skin fibroblasts. The fibroblasts are subjected to H₂O₂ stimulation and passaged at least five times after contacting with 10-HDA. A relative telomere length is determined, wherein the relative telomere length is determined by quantitative PCR as a ratio of a telomere repeat signal to a single-copy reference gene signal (T/S ratio). Relative to an untreated control skin fibroblast system subjected to the same H₂O₂ stimulation but not contacted with 10-HDA, the relative telomere length increases.

In some embodiments, the H₂O₂ stimulation is performed for three consecutive days, and each stimulation lasts two hours.

In some embodiments (e.g., Example 5), relative to an untreated control skin fibroblast system subjected to the same H₂O₂ stimulation but not contacted with 10-HDA, the relative telomere length increases by 34.6%.

The present disclosure is further explained below by way of Examples, which do not constitute a limitation of the present disclosure. It should be understood that these Examples are provided for illustration and not for limiting the scope of the present disclosure. Experimental methods without specified conditions are generally performed under conventional conditions. Reagents and medicines used in the Examples are commercially available products.

### Example 1: Preparation of 10-HDA Using an Engineered Yeast Strain

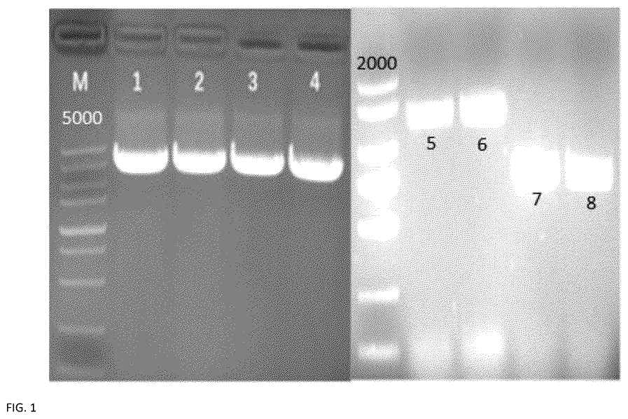

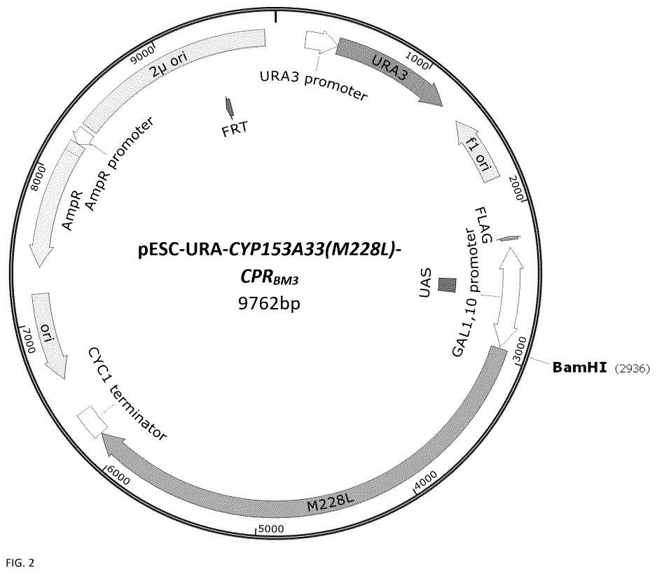

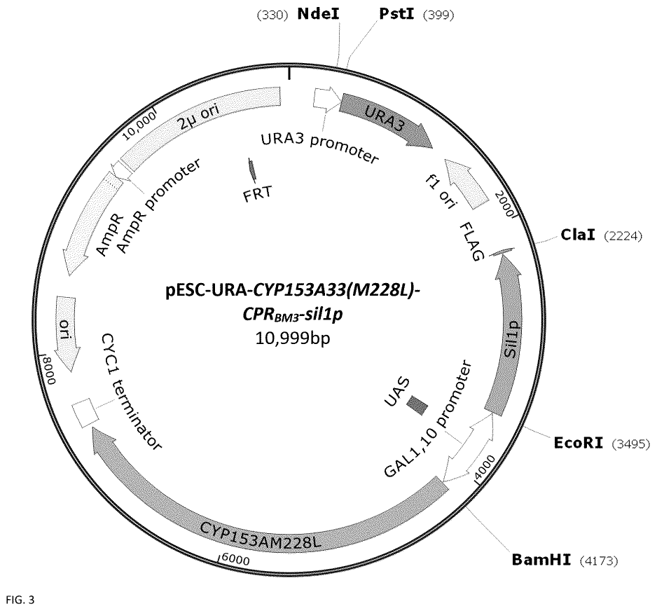

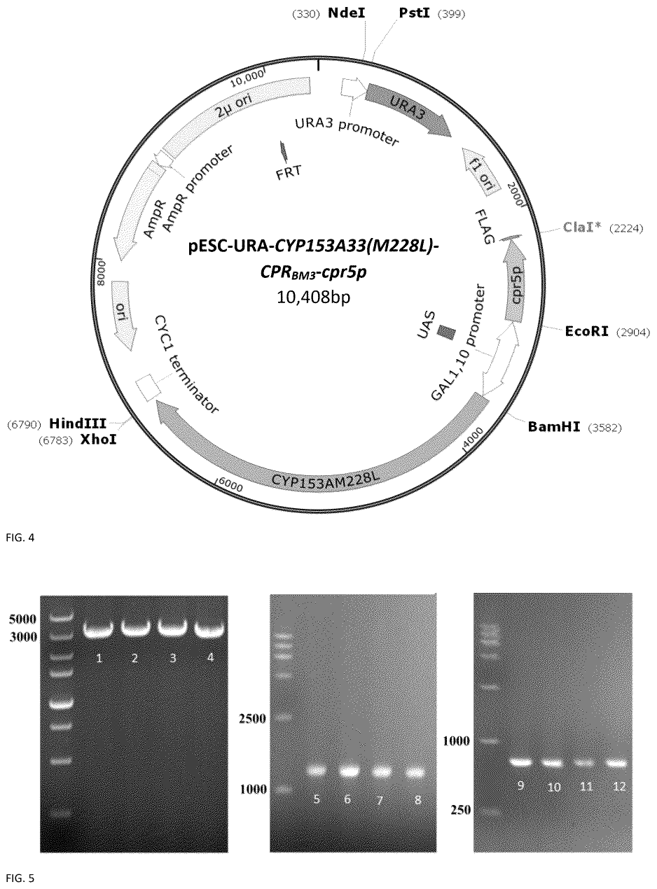

According to codon preference of S. cerevisiae, the CYP153A33(M228L)-CPR_{BM3} fusion gene was codon-optimized. The optimized nucleotide sequence is as shown in SEQ ID NO: 1. The nucleotide sequences of the auxiliary protein genes sil1p and cpr5p in S. cerevisiae are as shown in SEQ ID NO: 2 and SEQ ID NO: 3, respectively. The amplification products are shown in FIG. 1, where the target bands appear and are single. After full-gene synthesis, the gene was cloned into the S. cerevisiae expression vector pESC-URA between the BamHI and NotI restriction sites to obtain the recombinant expression vector pESC-URA-CYP153A33(M228L)-CPR_{BM3} (FIG. 2). The vector was then co-expressed with the auxiliary protein gene sil1p (SEQ ID NO: 2) and cpr5p (SEQ ID NO: 3) in S. cerevisiae, respectively. The auxiliary protein gene was cloned into the S. cerevisiae expression vector pESC-URA between the ClaI and EcoRI restriction sites to obtain the recombinant expression vectors pESC-URA-CYP153A33(M228L)-CPR_{BM3}-Sil1p (FIG. 3) and pESC-URA-CYP153A33(M228L)-CPR_{BM3}-cpr5p (FIG. 4), respectively. The recombinant expression vectors were transformed into E. coli DH5a for cultivation, and colony PCR identification was performed. The identification results are shown in FIG. 5, where the target bands appear and are single, indicating positive clones. DNA sequencing alignment confirmed that the recombinant sequences were correct. The recombinant expression plasmids were chemically transformed into S. cerevisiae BY4741, respectively. Recombinant transformants were screened on a uracil-dropout medium to obtain high-copy recombinant S. cerevisiae strains: Saccharomyces cerevisiae BY4741-pESC-URA-CYP153A33(M228L)-CPR_{BM3}, S. cerevisiae BY4741-pESC-URA-CYP153A33(M228L)-CPR_{BM3}-sil1p, and S. cerevisiae BY4741-pESC-URA-CYP153A33(M228L)-CPR_{BM3}-cpr5p.

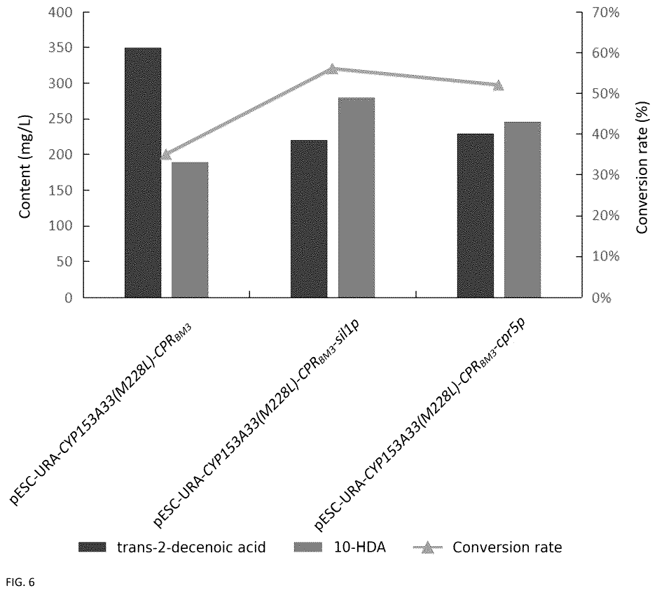

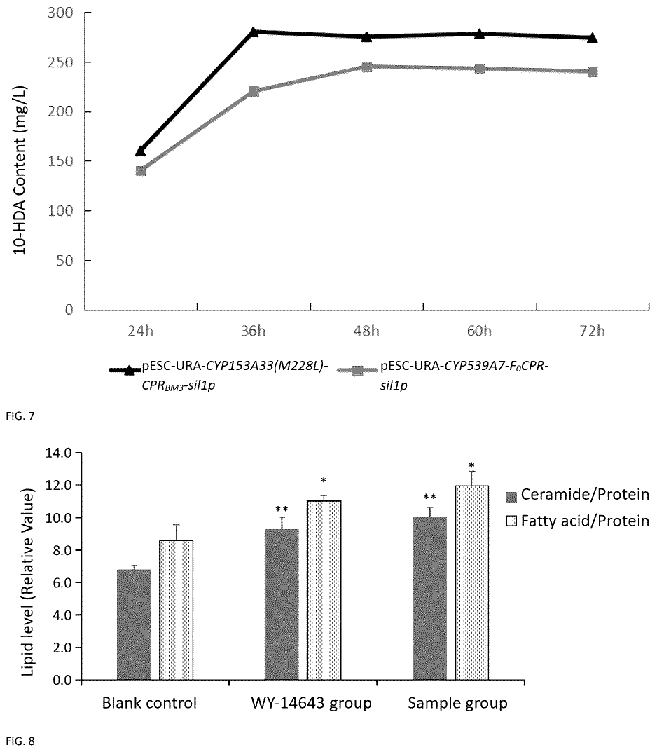

The method for producing 10-HDA using the above engineered strains is as follows: a single colony of an engineered yeast strain was inoculated into a uracil-dropout seed medium (basal medium YNB 6.7 g/L, uracil-dropout amino-acid mixture 1.29 g/L, and 2% (w/v) glucose) and cultured overnight with shaking at 30° C. and 200 rpm. The seed culture was inoculated into a pH 5.5 uracil-dropout fermentation medium (basal medium YNB 6.7 g/L, uracil-dropout amino-acid mixture 1.29 g/L, 4% (w/v) galactose, and 2 mM 5-ALA) at an initial OD₆₀₀ of 0.4. The culture was induced with shaking at 30° C. until OD₆₀₀ reached 1.0, and cells were collected. The collected cells were resuspended in the same uracil-dropout fermentation medium and supplemented with trans-2-decenoic acid to a final concentration of 0.5 g/L, and cultured at 30° C. for 36 hours. The culture was extracted with ethyl acetate, dried, and detected to obtain 10-hydroxy-2-decenoic acid. The yields and conversion rates of 10-hydroxy-2-decenoic acid produced by the three engineered S. cerevisiae strains are shown in FIG. 6. Recombinant expression of CYP153A33(M228L)-CPR_{BM3} can react with the substrate trans-2-decenoic acid to obtain 10-HDA, with a yield reaching 190 mg/L. Co-transformation and co-expression of the CYP153A33(M228L)-CPR_{BM3} fusion gene with the auxiliary protein gene sil1p and the auxiliary protein gene cpr5p in S. cerevisiae can effectively increase the yield of 10-HDA in the engineered yeast strain. With connection of the auxiliary protein gene sil1p to the protein, the 10-HDA yield can reach 280 mg/L. With connection of the auxiliary protein gene cpr5p to the fusion enzyme gene, the 10-HDA yield can reach 246 mg/L. In addition, as shown in FIG. 7, the engineered strain harboring a co-expression plasmid of CYP153A33(M228L)-CPR_{BM3} and the auxiliary protein gene sil1p in S. cerevisiae can reach the maximum conversion rate and product conversion amount after reacting with 0.5 g/L decenoic acid for 36 hours, which is superior to the engineered strain BY4741/pESC-URA-CYP539A7*-F0CPR*-sil1p described in CN202211449654.3.

### Example 2: Regulatory Effect of 10-HDA on Ceramides and Fatty Acids in a 3D Epidermal Model

The model was transferred to a 6-well plate pre-filled with 3.7 mL/well model culture medium and incubated for 24 hours in a CO₂ incubator (37° C., 5% CO₂). The positive control (PC) group contained WY-14643 (pirinixic acid) at a final concentration of 50 μM. In the sample group, 10-HDA was at a final concentration of 0.0625 mg/mL. After incubation, each model was cut into two halves. One half was used for protein measurement, and the other half was used for lipid extraction and placed in a 6-well plate (the model was stored at −80° C.). For lipid extraction, 1 mL trypsin was added to each well of the 6-well plate and incubated for 30 minutes in an incubator. Water was added on the lid of the 6-well plate. The model and the nylon membrane were separated with tweezers, and viscous material on the stratum corneum was peeled off with tweezers. Water on the stratum corneum was blotted dry with paper, and the stratum corneum was placed into a glass tube. 1 mL chloroform:methanol mixture (chloroform:methanol=1:1) was added to each glass tube. The tube was ultrasonicated in an ice bath for 30 minutes. The supernatant was transferred to a vial and evaporated to dryness. To the dried vial, 160 μL acetonitrile: isopropanol mixture (acetonitrile:isopropanol=1:1) was added, and 20 μL ceramide C12 internal standard solution was added (the ceramide internal standard solution having a concentration of 100 μg/mL, with the internal standard solvent being a 1:1 mixture of acetonitrile and isopropanol). The mixture was ultrasonicated for 10 minutes and shaken for dissolution for 30 minutes, transferred to a sample centrifuge tube, centrifuged at 12,000 rpm for 10 minutes, and 100 μL of the upper layer was transferred into a 250 μL insert. LC-MS detection was performed using a Thermo Fisher Orbitrap Q Exactive™ quadrupole-Orbitrap high-resolution mass spectrometer.

Mass spectrometry conditions were as follows: mass spectrometry analysis was performed using a quadrupole-Orbitrap mass spectrometer equipped with a heated electrospray ionization source. Ion source voltages for positive and negative modes were 3.7 kV and 3.5 kV, respectively. Capillary heating temperature was 320° C. Sheath gas pressure was 30 psi, and auxiliary gas pressure was 10 psi. Heated vaporizer temperature was 300° C. Both sheath gas and auxiliary gas were nitrogen. Collision gas was nitrogen at a pressure of 1.5 mTorr. Full MS scan parameters were: resolution 70,000; automatic gain control target 1×10⁶; maximum isolation time 50 ms; and m/z scan range 50-1500. The LC-MS system was controlled by Xcalibur 2.2 SP1.48 software, and data acquisition was also controlled by the software.

LC-MS detection parameters are shown in Table 1.

TABLE 1
LC-MS detection parameters

| Parameter | Condition/Setting | |
|---|---|---|
| Column | Waters UPLC BEH C8 | |
| | (1.7 μm, 2.1 mm × 100 mm) | |
| Flow rate | 0.26 | mL/min |
| Injection volume | 4 | μL |
| Elution | Gradient elution | |
| Run time | 16 | min |
| Column temperature | 40° | C. |

| | |
|---|---|
| Mobile phase | A (acetonitrile:water = 6:4, 0.1% formic acid, |
| | 5 mM ammonium acetate); |
| | B (isopropanol:acetonitrile = 9:1, 0.1% formic |
| | acid, 5 mM ammonium acetate) |

The LC-MS gradient program is shown in Table 2.

TABLE 2
LC-MS gradient program

| Time (min) | A (v/v %) | B (v/v %) |
|---|---|---|
| 0 | 100 | 0 |
| 2 | 70 | 30 |
| 12 | 30 | 70 |
| 12.5 | 5 | 95 |
| 13 | 0 | 100 |
| 14 | 0 | 100 |
| 14.1 | 100 | 0 |
| 16 | 100 | 0 |

The structural basis for the barrier-protective function of skin is the “brick-and-mortar” structure of the stratum corneum. The “brick” structure is mainly a protein envelope formed by crosslinking of transmembrane proteins and intramembrane proteins of corneocytes under the action of transglutaminase. The “mortar” is a lipid envelope formed by crosslinking extracellular lipids via esterification. The two are further crosslinked via esterification to form a highly sealed brick-and-mortar architecture that jointly resists the external environment and provides a permeability barrier. Ceramides and fatty acids are the primary lipids constituting the lipid envelope. LC-MS results (FIG. 8) show that treatment of the 3D epidermal model with 10-HDA can significantly promote expression of ceramides and fatty acids, with increases of 47.64% and 39.0%, respectively, thereby effectively restoring the sebum barrier.

### Example 3: Effect of 10-HDA on Damage Repair in Fibroblasts

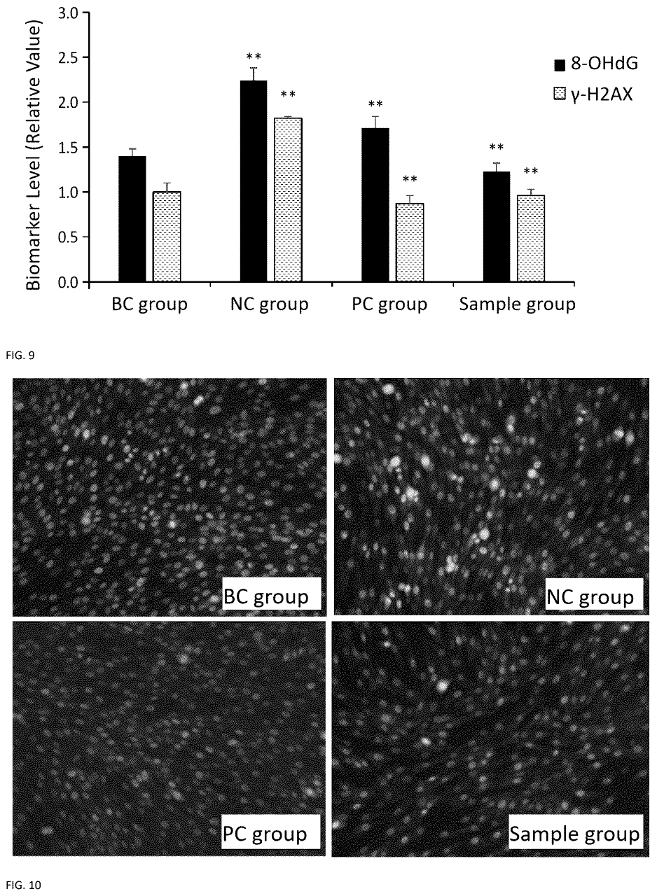

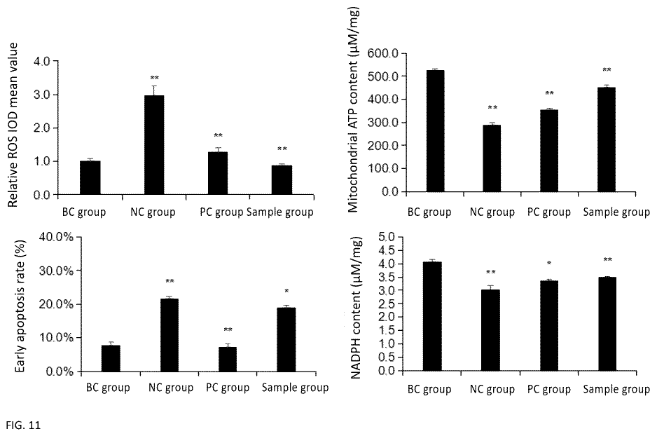

Fibroblasts were seeded into a 6-well plate at a seeding density of 2.2×10⁵ cells/well and incubated overnight in an incubator (37° C., 5% CO₂). When the confluence reached 40%-60%, 2 mL of culture medium containing a corresponding concentration of a test substance was added to each well according to different groups. The BC group was a blank control group without any drug intervention and was cultured only in 2 mL normal medium. The NC group was a negative control group without any drug intervention and was cultured only in 2 mL normal medium. The PC group was a positive control group containing VE at a final concentration of 7 μg/mL. The sample group contained 10-HDA at a final concentration of 0.25 mg/mL. After drug incubation for 24 hours, according to test groups, the NC group, PC group, and sample group were subjected to UV-A irradiation at a dose of 30 J/cm². After irradiation, the cells were further cultured for 24 hours in an incubator (37° C., 5% CO₂). After incubation, cell culture supernatants were collected and tested according to the operation instructions of an 8-OHdG kit. Meanwhile, cells in the culture plate were fixed with 4% paraformaldehyde for 24 hours, and immunofluorescence detection was performed using a DNA damage detection kit (γ-H2AX immunofluorescence). Images were acquired under a fluorescence microscope and analyzed using Image-Pro® Plus image processing software. As shown in FIG. 9, test results indicate that ultraviolet irradiation attacks the carbon atom at the 8th position of the guanine base in DNA molecules, leading to an increase in the DNA damage product 8-OHdG, and also causes phosphorylation of H₂AX Ser139 to generate the DNA damage product γ-H2AX. After intervention with 10-HDA, expression of both DNA damage products was inhibited, with inhibition rates of 45.09% for 8-OHdG and 47.25% for γ-H2AX, respectively. The immunofluorescence staining results in FIG. 10 further verify the above conclusion, namely that intervention with 10-HDA can effectively inhibit expression of the DNA damage product γ-H2AX in fibroblasts.

### Example 4: Effect of 10-HDA on Mitochondrial Function in Fibroblasts

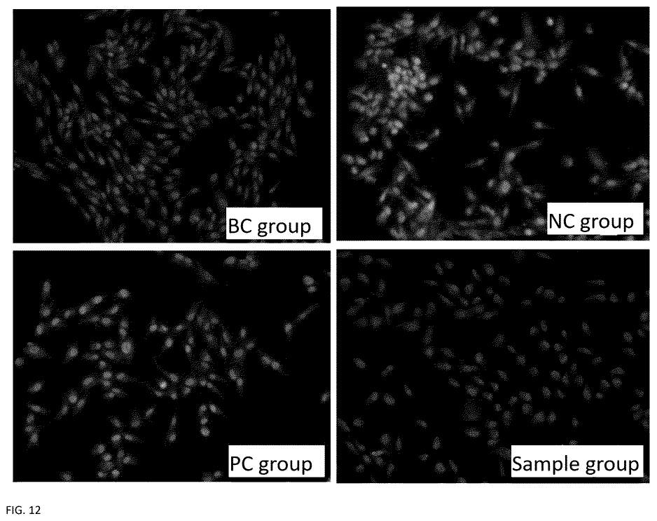

Fibroblasts were seeded into a 6-well plate at a seeding density of 2.2×10⁵ cells/well and incubated overnight in an incubator (37° C., 5% CO₂). When the confluence reached 40%-60%, 2 mL of culture medium containing a corresponding concentration of a test substance was added to each well according to different groups. The BC group was a blank control group without any drug intervention and was cultured only in 2 mL normal medium. The NC group was a negative control group without any drug intervention and was cultured only in 2 mL normal medium. The PC group was a positive control group containing VE at a final concentration of 7 μg/mL. The sample group contained 10-HDA at a final concentration of 0.25 mg/mL. After drug incubation for 24 hours, according to test grouping, the NC group, PC group, and sample group were subjected to UV-A irradiation at a dose of 30 J/cm². After irradiation, the cells were further cultured for 24 hours in an incubator (37° C., 5% CO₂). After incubation, mitochondrial functions were assessed according to instructions of a mitochondrial ROS detection kit, a mitochondrial ATP detection kit (Beyotime), a mitochondrial NADP⁺/NADPH detection kit (WST-8 method), and a JC-1 mitochondrial membrane potential detection kit. As shown in FIG. 11, test results indicate that ultraviolet irradiation significantly increased ROS content in fibroblasts and the apoptosis rate, and significantly decreased mitochondrial ATP and NADPH contents. After intervention with 0.25 mg/mL 10-HDA, ultraviolet irradiation-induced ROS and apoptosis were inhibited, with inhibition rates of 70.71% and 12.74%, respectively. In addition, ATP and NADPH were upregulated, with increases of 56.23% and 15.13%, respectively. Further, as shown in FIG. 12, intervention with 0.25 mg/mL 10-HDA can effectively inhibit generation of mitochondrial reactive oxygen species in fibroblasts, thereby improving antioxidant capacity.

### Example 5: Effect of 10-HDA on Telomere Length in Fibroblasts

Fibroblasts were seeded into a 6-well plate at a seeding density of 2.2×10⁵ cells/well and incubated overnight in an incubator (37° C., 5% CO₂). When the confluence reached 40%-60%, 2 mL of culture medium containing a corresponding concentration of a test substance was added to each well according to different groups. The BC group was a blank control group, wherein the cells were derived from back skin of children aged 7-8 years, and no drug intervention was performed during cultivation. The NC group was a negative control group, wherein the cells were derived from back skin of an adult aged 48 years. When the confluence reached 40%-60%, H₂O₂ stimulation was applied for three consecutive days, with each stimulation lasting 2 hours, and samples were collected for detection after continuous passaging for five passages. The PC group was a positive control group, wherein the cells were derived from back skin of an adult aged 48 years. When the confluence reached 40%-60%, H₂O₂ stimulation was applied for three consecutive days, with each stimulation lasting 2 hours. During the stimulation period, the cells were cultured in a medium containing cycloastragenol at a final concentration of 1 mg/mL. Samples were collected for detection after continuous passaging for five passages. The sample group cells were derived from back skin of an adult aged 48 years. When the confluence reached 40%-60%, H₂O₂ stimulation was applied for three consecutive days, with each stimulation lasting 2 hours. During the stimulation period, the cells were cultured in a medium containing 10-HDA at a final concentration of 0.25 mg/mL. Samples were collected for detection after continuous passaging for five passages. Genomic DNA (gDNA) was obtained using a genomic DNA extraction kit, and telomere length was determined by qPCR using gDNA as a template. The primer sequences used and the single-copy gene information are shown in Table 3.

TABLE 3
Primer sequences for telomere and single-copy gene
36B4 and product lengths

| | | | Product |
|---|---|---|---|
| Primer name | | Sequence | length |
| Telomere | Forward | 5′-CGGTTTGTTTGGGTTTGGGTTTGGGTTT | 76 bp |
| | | GGGTTTGGGTT-3′ (SEQ ID NO: 4) | |
| | Reverse | 5′-GGCTTGCCTTACCCTTACCCTTACCCTT | |
| | | ACCCTTACCCT-3′ (SEQ ID NO: 5) | |
| 36B4 | Forward | 5′-CAGCAAGTGGGAAGGTGTAATCC-3′ | 63 bp |
| | | (SEQ ID NO: 6) | |
| | Reverse | 5′-CCCATTCTATCATCAACGGTACAA-3′ | |
| | | (SEQ ID NO: 7) | |

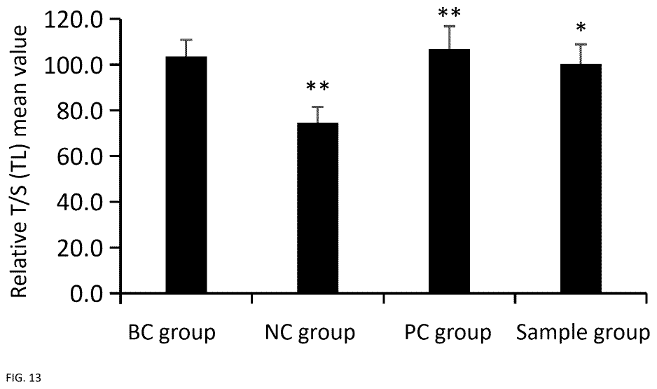

The results are shown in FIG. 13, test results indicate that telomere length in senescent cells of the NC group was significantly reduced and was only 72% of the telomere length of the BC group. The PC group significantly extended telomere length, with an increase of 43.1% relative to the NC group. After intervention with 10-HDA, telomere length was also significantly increased, with an increase of 34.6% relative to the NC group, indicating that 10-HDA can delay telomere shortening caused by cell division and replication during senescence, strengthen structural protection of chromosome ends, effectively prevent cells from losing proliferative activity and entering senescence, and thereby prevent skin aging.

### Example 6: Application of 10-HDA in Functional Skin Care Products

TABLE 4
Formulation of a skin care lotion

Content (wt %)

| No. | Ingredient | Blank Example | Example |
|---|---|---|---|
| 1 | Butylene glycol | 4.0 | 4.0 |
| 2 | C12-20 alkyl glucoside | 2.0 | 2.0 |
| 3 | Carbomer | 0.15 | 0.15 |
| 4 | Tromethamine | 0.12 | 0.12 |
| 5 | Dimethicone | 1.0 | 1.0 |
| 6 | Isononyl isononanoate | 1.0 | 1.0 |
| 7 | 1,2-Hexanediol | 0.4 | 0.4 |
| 8 | p-Hydroxyacetophenone | 0.4 | 0.4 |
| 9 | Disodium EDTA | 0.05 | 0.05 |
| 10 | 10-HDA | — | 0.25 |
| 11 | Purified water | q.s. to 100 | q.s. to 100 |

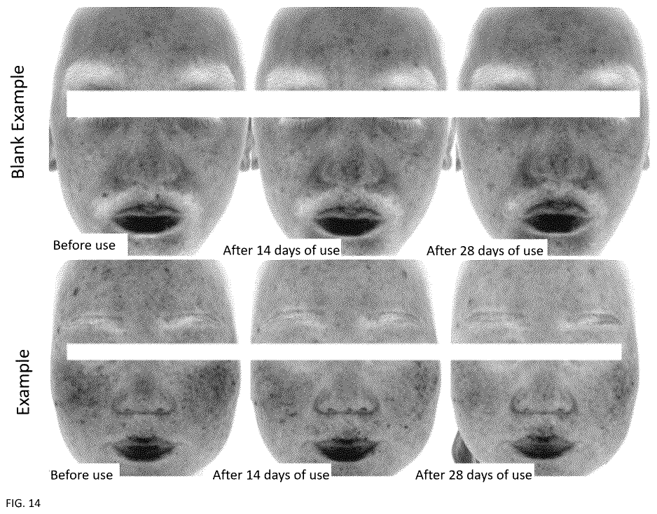

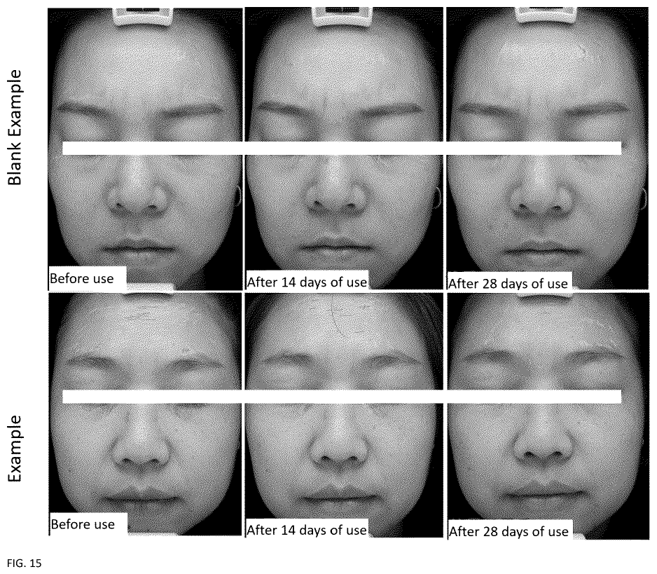

10-HDA was added into a skin care base lotion according to the formulation and content shown in Table 4 and mixed at room temperature for 5 minutes to obtain a skin care lotion. The skin care lotion was preferably used for cosmetics having barrier-repair and anti-aging efficacy. A population of 35-year-old sensitive-skin subjects with impaired barrier was selected. Testers continuously used the lotion for 14 days and 28 days. Facial photographs of subjects were taken using VISIA. Changes in red-area parameters before and after use were used to evaluate beneficial effects of 10-HDA-containing lotion on the skin barrier, and changes in wrinkle counts before and after use were used to evaluate beneficial effects of 10-HDA-containing lotion on skin aging. The results show that, after 28 days of using 10-HDA-containing lotion, red-area parameters were significantly improved (FIG. 14), and both the number and depth of wrinkles were reduced (FIG. 15), indicating that 10-HDA has good barrier-repair and anti-aging efficacy.

The above describes only preferred embodiments of the present disclosure and is not intended to limit the present disclosure. Various modifications and changes can be made by a person of ordinary skill in the art. Any modifications, equivalent substitutions, improvements, etc. made within the spirit and principles of the present disclosure shall be included within the scope of protection of the present disclosure.

1. An in vitro method for improving at least one skin-related biomarker of a skin-related in vitro system, comprising:
(a) contacting the skin-related in vitro system with 10-hydroxy-2-decenoic acid (10-HDA), wherein the skin-related in vitro system is selected from (i) a 3D epidermal model and (ii) skin fibroblasts; and
(b) determining a level of the at least one biomarker, wherein:
(i) when the skin-related in vitro system is the 3D epidermal model, the at least one biomarker is a stratum corneum lipid level, and, relative to an untreated control system not contacted with 10-HDA, the contacting causes the stratum corneum lipid level to increase; and
(ii) when the skin-related in vitro system is the skin fibroblasts, the at least one biomarker is selected from (A) a DNA damage marker level, (B) a mitochondrial function metric, and (C) a relative telomere length, and, relative to an untreated control system not contacted with 10-HDA, the contacting causes at least one of:
(1) the DNA damage marker level to decrease;
(2) the mitochondrial function metric to improve, the improvement comprising a decrease in mitochondrial ROS level and/or early apoptosis rate, and/or an increase in mitochondrial ATP content and/or cellular NADPH content; and
(3) the relative telomere length to increase.

2. The method of claim 1, wherein the skin-related in vitro system is the 3D epidermal model, and the stratum corneum lipid level comprises one or more of: total ceramide, ceramide/protein, total fatty acid, and fatty acid/protein.

3. The method of claim 2, comprising extracting lipids from a stratum corneum sample and quantifying the stratum corneum lipid level by liquid chromatography-mass spectrometry (LC-MS).

4. The method of claim 3, wherein, when the 3D epidermal model is contacted with 10-HDA, a final concentration of 10-HDA in a 3D epidermal model culture system is 0.0625 mg/mL.

5. The method of claim 4, wherein, relative to an untreated control 3D epidermal model system not contacted with 10-HDA:
(i) total ceramide increases by 35.37% and/or ceramide/protein increases by 47.64%; and/or
(ii) total fatty acid increases by 27.51% and/or fatty acid/protein increases by 39.0%.

6. The method of claim 1, wherein the skin-related in vitro system is the skin fibroblasts.

7. The method of claim 6, wherein, after incubating the skin fibroblasts with 10-HDA for 24 hours, the skin fibroblasts are subjected to UV-A irradiation at a dose of 30 J/cm² and cultured for an additional 24 hours after irradiation.

8. The method of claim 7, wherein a final concentration of 10-HDA in a skin fibroblast culture system is 0.25 mg/mL.

9. The method of claim 8, wherein the at least one biomarker comprises a DNA damage marker level, and the DNA damage marker level comprises an 8-hydroxy-2′-deoxyguanosine (8-OHdG) level and/or a γ-H2AX level.

10. The method of claim 9, wherein, relative to an untreated control skin fibroblast system subjected to the UV-A irradiation and not contacted with 10-HDA, the 8-OHdG level is reduced by 45.09%.

11. The method of claim 9, wherein, relative to an untreated control skin fibroblast system subjected to the UV-A irradiation and not contacted with 10-HDA, the γ-H2AX level is reduced by 47.25%, and the γ-H2AX level is measured by γ-H2AX immunofluorescence.

12. The method of claim 8, wherein the at least one biomarker comprises a mitochondrial function metric, and the mitochondrial function metric comprises one or more of: mitochondrial ROS level, mitochondrial ATP content, cellular NADPH content, and early apoptosis rate.

13. The method of claim 12, wherein, relative to an untreated control skin fibroblast system subjected to the UV-A irradiation and not contacted with 10-HDA: mitochondrial ROS level is reduced by 70.71%, and/or mitochondrial ATP content is increased by 56.23%, and/or cellular NADPH content is increased by 15.13%, and/or early apoptosis rate is reduced by 12.74%.

14. The method of claim 6, wherein the at least one biomarker comprises a relative telomere length, and the fibroblasts are subjected to H₂O₂ stimulation and passaged at least five times after contacting with 10-HDA.

15. The method of claim 14, wherein the relative telomere length is determined by quantitative PCR as a T/S ratio, and the H₂O₂ stimulation is performed for three consecutive days, with each stimulation lasting two hours.

16. The method of claim 15, wherein a final concentration of 10-HDA in a skin fibroblast culture system is 0.25 mg/mL.

17. The method of claim 16, wherein, relative to an untreated control skin fibroblast system subjected to the H₂O₂ stimulation and not contacted with 10-HDA, the relative telomere length is increased by 34.6%.
